(19) United States
(12) Patent Application Publication
(10) Pub. No.: US 2011/0282247 A1
(43) Pub. Date: Nov. 17, 2011

(54) Novel Ovulation Predictor Device

(76) Inventors: Helen Denise, Newark, NJ (US); Russ Case, Oviedo, FL (US)

(21) Appl. No.: 13/076,728

(22) Filed: Mar. 31, 2011

Related U.S. Application Data
(60) Provisional application No. 61/319,355, filed on Mar. 31, 2010.

(51) Int. Cl. A61B 5/103; G01N 21/01

(52) U.S. Cl. 600/588; 356/244; 382/128

(57) ABSTRACT

The invention is a hand held ovulation predictor device, which includes an ovulation predictor device body, an optical subassembly containing one or more aspheric lenses, an electronics assembly, a battery compartment, a light source and, optionally, a cover. The preferred embodiment of this invention is one in which the one or more lenses are aspheric.

### CLAIM OF PRIORITY

This application claims the priority of U.S. Ser. No. 61/319,355 filed on Mar. 31, 2010, the contents of which are fully incorporated herein by reference.

### FIELD OF THE INVENTION

The present invention relates to devices for predicting ovulation in animals, in particular to hand held ovulation predictors.

### BACKGROUND OF THE INVENTION

Devices for predicting ovulation in animals are well known in the art. Women, especially busy women, require an accurate and portable device to predict ovulation. Knowing when she will ovulate can help predict fertile times in a female's cycle and assist the woman in becoming pregnant or avoiding pregnancy. This invention utilizes a unique device to predict ovulation based on hormonal changes which occur in a female during ovulation and the resultant change in the make-up of her bodily fluids during this important time.

Papanicolaou (PAPANICOLAOU, G. N.: Ibid. 51: 316, 1946.) in 1945 described the crystals which are formed when a drop of cervical mucus is placed on a slide and allowed to dry. This characteristic crystallization was also described by Rydberg and Madsen (RYDPERG, E. AND MADSEN, V.: Acta obst. & gynec.scandinay., 28: 386, 1948.) in 1948. Rydberg also reported the chemical composition of the crystals. The crystals were found to be common salt, and the crystal formations were due to the presence of mucin. Zondek (ZONDEK, B. AND ROZIN, S.: Obst. & Gynec., 3: 463, 1954.) has shown that the crystallization is not specific for cervical mucus because the same phenomenon appears in all mucus secretions and most body fluids.

This device utilizes the phenomenon known as “ferning” wherein a specimen of dried fluid sample produces crystals of a particular characteristic which is indicative of ovulation.

Several devices for ovulation prediction are known.

U.S. Pat. No. 6,793,886 is directed to a kit for the detection of characteristics and parameters of body fluids such as saliva, urine and cervical mucus for identifying fertility comprising a set of flat plate-shaped supports for samples of said body fluids and a viewer provided with an enlargement means. Each of said flat plate-shape supports for body fluid presents a shallow basin or trap with a convex bottom with a raised rim, and is equipped with locking fins for coupling with the viewer.

US 2003/0179446 is directed to a portable microscopic visualization tube for determining ovulation from saliva assay. It has a microscopic lens module, a beam tube, an electric powered LED mechanism, and a tube cap, and the LED mechanism includes a button battery seat, characterized in that the mounting position of the edge of the button seat and the inner wall of the beam tube is correspondingly formed into recessing block or protruding block such that the entire LED mechanism can be withdrawn from the beam tube to replace the battery.

US 2006/0018043 is directed to a portable handheld fertility/ovulation tester that uses ambient light. A sample holding frame and adjustable lens assembly is inserted into a light chamber in the bottom of the tester. An aperture in the bottom of the chamber is aligned with a microscope lens assembly and is sized to provide ambient light for the microscope lens assembly. The aperture may also have an optional light gathering lens to increase illumination. The adjustable lens assembly is threaded into a sample plate frame having a transparent sample plate. The microscope lens assembly is removably mounted onto the light chamber such that when the fertility ovulation tester must be held with the aperture pointed towards an ambient light source in order to observe the sample.

U.S. Pat. No. 7,369,331 provides a fixed focus ovulation tester comprising an inner casing, having a top and a bottom end; a controllable illuminating assembly located inside the inner casing and near the bottom end of the inner casing and being sealed at the bottom by a bottom face plate and a fixed focus eye piece assembly having a bottom inner portion for placing a biological specimen and a top outer portion for viewing the specimen being removably located at the top end of the inner casing.

In the currently available devices, aspherical lenses are not utilized. In the case of a spherical lens, light enters the lens both along its axis and distant from the axis. This creates an aberration producing a blurry image around the perimeter of the image field. This is noticeable with the human eye, causing the user to attempt to refocus the image, and is more particularly noticeable when utilizing electronic imaging. Further, light entering these lenses antiparalel to the axis produces a coma aberration and results in a hazy image, especially when viewing crystals spread across a finite surface.

Further, the current devices utilize either ambient light or one or more battery compartments without the improvements in the current invention which prevent erosion of the battery compartment, and result in loss of power to the device early on in the device's life.

There is a need for an inexpensive, compact and portable ovulation predictor device that can fit in the hand and which has reduced aberration and reliable, constant power.

### SUMMARY OF THE INVENTION

The invention is a hand held ovulation predictor device 100 for women, which includes an ovulation predictor device body 101, an optical subassembly 300 containing one or more aspheric lenses 303, an objective mount 301 and a focus ring 302 being movably connected to the ovulation predictor objective mount 301. Further the aspheric lenses 303 are held within the objective mount 301 and the focus ring 302 is held within the objective mount 301. The objective plate 304 is intimately connected to the objective mount 301. The objective plate receives a specimen, when it is pulled out of the body 101 along with the optical assembly 400. Further, the objective mount 301 is closely connected to the body 101, and the body is removeably connected to the battery compartment 401 and the base 402 fits into the battery compartment, as do all the pieces of the electronics assembly 400.

The ovulation predictor device 100 also comprises an electronics assembly 400 which fits into the battery compartment 401. The electronics assembly 400 includes the battery compartment 401, a switch 403 one or more printed circuit boards 404 one or more contacts 405, a resistor 407 and a light 406 source. In one embodiment, the device has an elliptical shape. In a preferred embodiment, the device is tubular in shape.

The switch 403 may be placed anywhere in the ovulation predictor device 100 and may have any type of switch 403 known in the art, including an on-off mechanical switch 403, a spring loaded switch 403, a momentary—on push button and the like. Another embodiment of this invention is one in which the ovulation predictor device body 101 optionally includes a mechanical actuator which is spring loaded and activated by removal of a protective cover 102.

In another embodiment, there are one or more lenses 303. The lenses 303 are made from any material known on the art, including glass, plastics or resins. The lens shape is selected from concave, convex, plano-convex, spherical or aspheric. In a preferred embodiment, the one or more lenses 303 are aspheric. In another preferred embodiment, the optical subassembly 300 provides a broad optical field of view and a sharper image, not present in other ovulation predictors known in the art.

Therefore, the present invention succeeds in conferring the following, and other not mentioned, desirable and useful benefits and objectives.

It is an object of the present invention to provide a portable hand-held ovulation predictor device.

It is another object of the present invention to provide an enlarged optical field of view and a sharper image.

Yet another object of the present invention is to provide a convenient light activation switch.

Still another object of the present invention is to provide an ovulation predictor device that includes a battery compartment that is capable of providing long-term and reliable power by eliminating galvanic corrosion to the battery contacts.

Yet another object of the present invention is to provide a device that may be able to be powered by DC or AC currents, through storage of mechanical actions converted to an electrical energy, via a piezoelectric source, by capturing electrical fields present in the environment, or by utilizing panels that convert solar light and storing it.

Still another object of the present invention is to provide a method of determining ovulation in a female from a specimen of her bodily fluid, such as saliva, allowing it to dry, and observing a particular “ferning” pattern utilizing the ovulation predictor device 100 described herein.

### BRIEF DESCRIPTION OF THE DRAWINGS

FIG. 1 is a side exploded view of parts of an embodiment of the present invention.

FIG. 2 is aside exploded view of parts of an embodiment of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

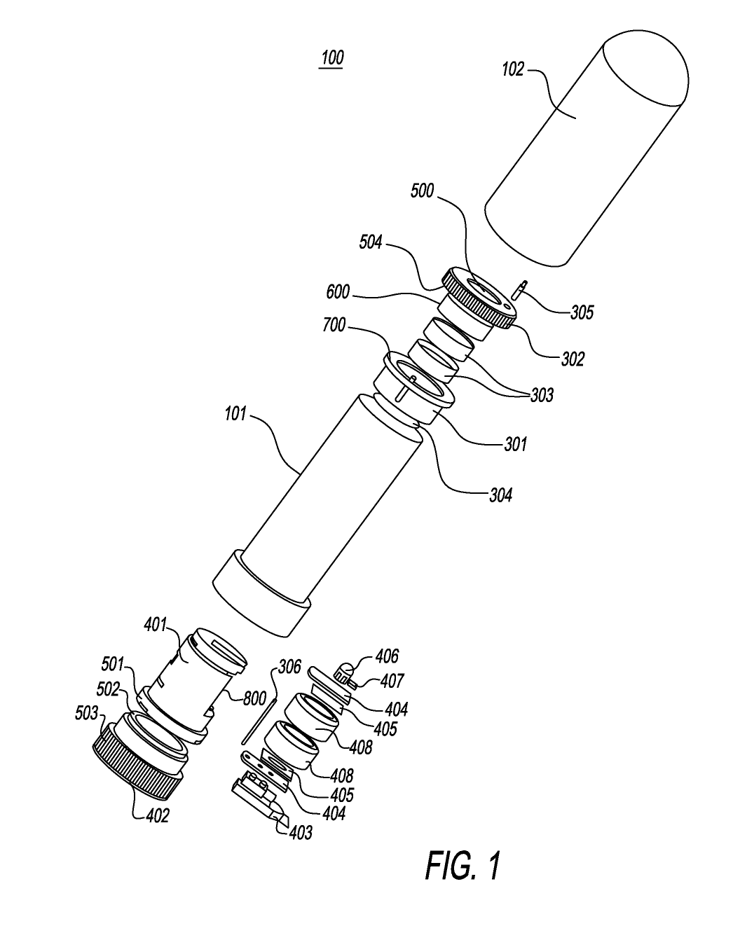

FIG. 1 is a side exploded view of a preferred embodiment of the present invention. Shown ovulation predictor device body 101, an optical subassembly 300 containing one or more aspheric lenses 303, an objective mount 301 and a focus ring 302 being movably connected to the ovulation predictor objective mount 301. Further the aspheric lenses 303 are held within the objective mount 301 and the focus ring 302 is held within the objective mount 301. The objective plate 304 is intimately connected to the objective mount 301. The objective plate receives a specimen, when it is pulled out of the body 101 along with the optical assembly 400. Further, the objective mount 301 is closely connected to the body 101, and the body is removeably connected to the battery compartment 401 and the base 402 fits into the battery compartment, as do all the pieces of the electronics assembly 400.

The ovulation predictor device 100 also comprises an electronics assembly 400 which fits into the battery compartment opening 800. The electronics assembly 400 includes the battery compartment 401, a switch 403 one or more printed circuit boards 404 one or more contacts 405, a resistor 407 and a light 406 source. In one embodiment, the device has an elliptical shape. In a preferred embodiment, the device is tubular in shape.

The switch 403 may be placed anywhere in the ovulation predictor device 100 and may have any type of switch 403 known in the art, including an on-off mechanical switch 403, a spring loaded switch 403, a momentary—on push button and the like. Another embodiment of this invention is one in which the ovulation predictor device body 101 optionally includes a mechanical actuator which is spring loaded and activated by removal of a protective cover 102.

In another embodiment, there are one or more lenses 303. The lenses 303 are made from any material known on the art, including glass, plastics or resins. The lens shape is selected from concave, convex, plano-convex, spherical or aspheric. In a preferred embodiment, the one or more lenses 303 are aspheric. In another preferred embodiment, the optical subassembly 300 provides a broad optical field of view and a sharper image, not present in other ovulation predictors known in the art.

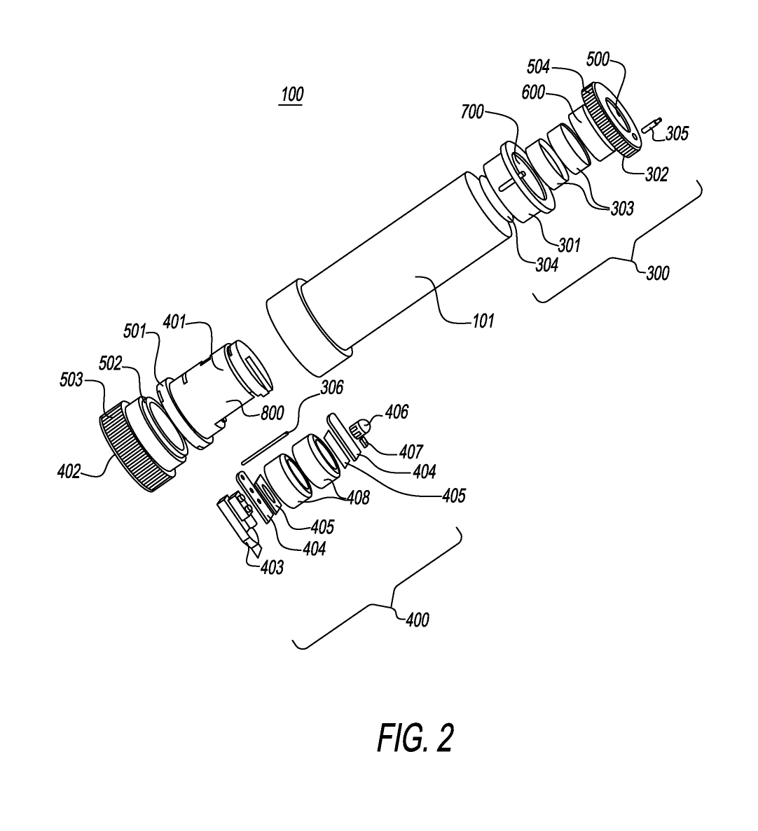

In FIGS. 1 and 2, we show the ovulation predictor device body 101, an optical subassembly 300, an electronics assembly 400, a battery compartment 401, the battery compartment opening 800, a base 402, a light source 406 and, optionally, a cover 102. In addition, the optical subassembly is made of an objective plate 304, an objective mount 301, one or more lenses 303, a focus ring 302 and a focus ring lock pin 305.

The ovulation predictor device 100, has an electronics assembly consisting of a battery compartment 401, a base 402, a switch 403, a printed circuit board 404, one or more contacts 405, a light 406 source a resistor 407, and one or more batteries 408.

In preferred embodiments, the light 406 source is a LED. In another preferred embodiment one or more lenses are aspherical. An aspherical lens enlarges the optical field and reduces aberration and blurring of the image as compared to a spherical lens.

In the ovulation predictor device the battery compartment 401 has one or more contacts 405, preferably a positive and a negative contact 405. The contact is intimately in contact with the batteries. In a preferred embodiment, the battery compartment contacts 405 are manufactured from nickel. More preferably, the battery compartment contacts 405 have dimpled or raised surfaces.

The ovulation predictor device 100 and the body 101 may have an elliptical shape, but may any other shape, including round, tubular or cylindrical, spherical shape, square, triangular, rectangular and the like. FIGS. 1 and 2 show the tubular or cylindrical shape, which, for the purposes of this invention, are synonymous. The body 101 may have a smooth or an uneven surface. In one embodiment the body 101 may have may have ribs, finger indentations, or other shapes or indentations which are useful in achieving a good grip and to hold the ovulation predictor device 100 as well as to assist in gripping the focus ring 302.

The body 101 may be of any length or width. In a preferred embodiment the ovulation predictor device 100 fits in the palm of a human hand or in a woman's purse. In one embodiment, the diameter is about 0.8 inches and the length is about 2.5 inches. In another embodiment, the diameter and lengths may have different ranges as in elliptical or spherical shapes. For example, the diameter may range from about 0.025 to about 5.5 inches. The length may be equal to or greater than or less than the diameter. In other embodiments, the length may be about 0.25 inches to about 5.5 inches.

The ovulation predictor device 100 can be any shape and in a preferred embodiment is cylindrical. In another embodiment, the ovulation predictor device 100 may be approximately the size of a lipstick dispenser (approximately 0.81 inches in diameter by approximately 2.63 inches long). In another embodiment ovulation predictor device 100 may be between about 2 to about 4 inches long and between about 0.5 and 3 inches wide. In another embodiment ovulation predictor device 100 may be between about 1 to about 3 inches long and between about 0.25 to 2 inches wide. In another embodiment the ovulation predictor device 100 may be substantially elliptical. In another embodiment, the ovulation predictor device 100 is substantially uniform in length and width. In another embodiment, the ovulation predictor device 100 is not uniform in shape and width.

The optical subassembly 300 is made of an objective mount 301, a focus ring 302, one or more lenses 303, and objective plate 304, and a focus ring lock pin 305. Preferably the focus ring 302 has a focus ring top 500 which is a clear, see-through top from which to view a specimen. The specimen is preferably a mammalian specimen, which is usually a fluid from a mammal's body and may be any one of saliva, urine, mucus, vaginal secretions and the like. In one embodiment, the mammal is a human. In another embodiment, the human is a female. The specimen is placed on the objective plate 304, allowed to dry and then observed for the presence of ferning through the focus ring top 500. The focus ring top, may also be a magnifying lens which assists the user in viewing the specimen.

Preferably, the focus ring 302 fits into the objective mount 301, both having threads, threads on the focus ring 600 which slide over threads on the inside receptacle of the objective mount 700, and allow the user to focus his or her view of the specimen. The focus ring 302 has a focus ring top 500 from where to view a specimen, a grip 504 to allow a user to adjust the focus. The lenses 303 are in intimate contact with the objective mount 301, and I a preferred embodiment the objective mount 301 acts as a receptacle for the one or more lenses 303 and for the threaded portion 600 of the focus ring 302.

In a preferred embodiment the specimen is saliva. In another preferred embodiment, the user focuses on ferning created by crystals formed by the dried specimen when using the ovulation predictor device 100.

In one embodiment the lens 303 diameter is approximately 0.31 inches. In another embodiment, the diameter is from about 0.1 to about 1.5 inches in diameter. The lens may be produced by injection molding or diamond turning of base materials, or by any other method known in the art. The lens 303 base material or materials may be glass, plastics, resins and other materials disclosed herein and those known in the art.

The lens 303 may be any shape and there may be one or more lenses 303 present. The lens shape is selected from one or more concave, convex, plano-convex, spherical or aspheric. In, a preferred embodiment, the lens 303 is aspheric. In another preferred embodiment there are two lenses 303. In a most preferred embodiment, there are two aspheric lenses 303. The objective mount 301 and the focus ring 302 may be made out of the same materials as the lens 303. The objective mount 301 and the focus ring 302 may be made out of different materials as the lens 303.

In another embodiment the lens may have any focal length. In one embodiment, the focal length is about 0.1 inches. In another embodiment, the focal length is from about 0.2 to about 0.1 inches.

By using the aspheric lens 303 or lenses 303 in the preferred embodiment, the viewing image and therefore the accuracy of the device is improved over other devices. As discussed above, the aspheric lens 303 in this optical subassembly 300 provides a broad optical field of view and a sharper image, not present in other ovulation predictors known in the art. Accuracy in determining “ferning” in a specimen is enhanced by this improved optical assembly and provides for a more reliable result for the user. Ferning is well known in the art and means crystallization produced by a specimen, wherein crystal precipitate out of solution upon drying of the specimen, and thereby produce a dry, crystallized specimen that resemble fern leaves. A better view of ferning when it occurs, increases the chances of a woman knowing when she is ovulating, and may increase her chances of becoming pregnant. This is a substantial improvement over other prediction devices utilizing the presence of ferning. Further, this method might be used by a physician in his or her office for fast and economical determination of ovulation.

The ovulation predictor device 100 may also assist in screening pregnant woman for premature rupture of membranes (PROM) during pregnancy, and reduce the risk of undiagnosed high risk pregnancies. (Davidson K M. Detection of premature rupture of the membranes. Clin Obstet Gynecol. 1991; 34:715-22). One of the most common complications of preterm PROM is early delivery. The latent period, which is the time from membrane rupture until delivery, generally is inversely proportional to the gestational age at which PROM occurs. For example, one large study of patients at term revealed that 95 percent of patients delivered within approximately one day of PROM, whereas an analysis of studies evaluating patients with preterm PROM between 16 and 26 weeks' gestation determined that 57 percent of patients delivered within one week, and 22 percent had a latent period of four weeks. When PROM occurs too early, surviving neonates may develop sequelae such as malpresentation, cord compression, oligohydramnios, chorioamnionitis, abrupto placentae, necrotizing enterocolitis, neurologic impairment, intraventricular hemorrhage, respiratory distress syndrome and antepartum fetal death. Furthermore, numerous mothers will deliver within one week of PROM. Numerous risk factors are associated with preterm PROM. Black patients are at increased risk of preterm PROM compared with white patients. (Savitz D A, Blackmore C A, Thorp J M. Epidemiologic characteristics of preterm delivery: etiologic heterogeneity. Am J Obstet Gynecol. 1991; 164:467-71). Other patients at higher risk include those who have lower socioeconomic status, are smokers, have a history of sexually transmitted infections, have had a previous preterm delivery, have vaginal bleeding, or have uterine distension (e.g., polyhydramnios, multifetal pregnancy). (American College of Obstetricians and Gynecologists. Premature rupture of membranes. Clinical management guidelines for obstetrician-gynecologists. ACOG practice bulletin no. 1. Int J Gynaecol Obstet. 1998; 63:75-84). The improved optical subassembly 300 provided by utilization of the one or more aspheric lenses 303 of this invention, can assist the physician by improving the accuracy of such a diagnosis and thereby prevent fetal morbidity and mortality.

The electronics subassembly 400 consists of a battery compartment 401, a base 402, a switch 403, one or more printed circuit boards 404, one or more contacts 405, a light 406, a resister 407 and one or more batteries 208. The battery compartment 401 holds the components of the electronics assembly 400 in the battery compartment opening 800 which is a cut-out portion for holding the items which make up the electronics assembly. The electronics subassembly 400 may be any shape or made from any materials know to those skilled in the art, or those materials described herein. It may be made of the same material or different material than the body 101, and include any material described herein.

The body 101 may be any shape or size. In a preferred embodiment, it is made to be in intimate contact with objective mount 301 on one side and the battery compartment 401 on the other side. The lip 501 on the lower end of the battery compartment fits into the base 401. The base 403 has a grip 503 to assist the user in activating the device.

In one embodiment, the objective mount 302 is located on top of the device. In another embodiment, the objective mount 302 is located along the side of the device. In still another embodiment, the objective mount 302 may be located in the bottom of the device. The objective mount 302 may be a simple open end through which the use observes the specimen. In another embodiment, the focus ring 302 is aided by a magnification device. The magnification device may be a magnification lens. In still another embodiment, the user may interface electronically, the ovulation prediction device 100 with an electronic device such as a computer or a camera, and view the image with the assistance of a camera or monitor. These electronic devices may act as magnification devices for the focus ring 302. In another embodiment, the objective mount 302 image is collected and stored on a disc, such as a mini-disc or on a computer or camera's disc or drive.

The objective plate 304 is used to hold the sample and is in intimate contact with the objective mount 301. The objective plate 304 may be made preferably of glass, but may be made of any translucent material sufficient to permit the passage of light and permit the user to view dried specimens, such as saliva. In another embodiment, the specimens may be from any bodily fluid including, but not limited to saliva, tears, urine, vaginal secretions or sweat. In one embodiment, the specimen is from a mammal. In another embodiment, the mammal is a human. In still another embodiment, the mammal is a female. The objective plate 304 can be placed anywhere in or on the ovulation predictor device 100 where it can be illuminated and viewed by the user. In a preferred embodiment, the objective plate 304 is placed above the light 406 source. In another embodiment the objective plate 304 is located below the light 406 source.

The body 101 may also be of any shape or size. In one embodiment the body 101 is a continuous smooth surface. In another embodiment the body 101 shape and manufacturing material are as described herein. The body 101 may be interrupted at some point to provide for insertion of a switch 403. In an alternative embodiment, the body 101 surface may be prepared by or coated with a conducting material, such as is used in metal oxide semiconductor field effect transistor technology (MOSFET), and thereby also acts as the on/off switch of the ovulation predictor device 100.

The ovulation predictor device 100 optionally contains a carrying case designed to protect the device when carrying the device, for example in a purse or luggage. In a preferred embodiment, the ovulation predictor is a kit comprising the ovulation prediction device 100, a holder and instructions. The instructions may be written or in electronic format or may utilize more than one means of communication of instructions and educational materials. These instructions and education materials may be in one or more languages. The ovulation predictor device 100 may be supplies in a kit comprising the ovulation predictor device of claim, instructions on how to use the device and, optionally a carrying case. A kit may also comprise the ovulation predictor device, instructions on how to use the device, a carrying case and a cloth with which to clean the objective plate. In a most preferred embodiment, the kit comprises the ovulation predictor device, a multi-language book with instructions, an educational interview on compact disc, a discrete carrying case, a microfiber cleaning cloth and a fertility chart.

The instructions may contain information on how to utilize the ovulation predictor device 100 and other useful or educational material.

In another embodiment, the ovulation predictor device 100 fits ergonomically in the human hand. The ovulation predictor device 100 may be made of rubber, foam, neoprene and may be smooth or may have raised bumps or a by kind of texture. The ovulation predictor device 100, its body 101, electronics assembly 400 and optical subassembly 300 may be made from any material, including but not limited to: plastics and resins including but not limited to plastic, rubber, foam, silicone, ABS, Polycarbonate, Noryl™, PVC, Polystryrene, ABS/PVC, PVC/Acrylic, Polysulfone, Acrylic, Polyethylene, Kydex™, PETG; glass, including but not limited to fiberglass, borosilicate, or quartz; wood; metals, including but not limited to nickel, iron, tin, aluminum, copper; rubber including but not limited to natural rubber, SBR, Isoprene rubber, Butadiene rubber, and Chloroprene rubber; or any combinations or composites of these materials or other materials and new materials that may be manufactured in the future. The parts to the ovulation predictor device 100 may be manufactured using identical or different materials. The parts may also be manufactured using injection molding techniques known to those skilled in the art.

Turning to FIG. 2, the body 101 may optionally slide into a protective cover 102. The light 406 may have a limiting resistor 407. The light 406 may be an LED and the LED may be any color. In a preferred embodiment, the LED is green. The LED may emit any range of light in the spectrum, preferably in the spectrum of visible light. In one embodiment, the spectrum is between about 525 to about 555 nm. The light may be filtered by a filtering device known to those in the art. The light may optionally be polarized by methods known to those in the art. Optionally, the light may be manipulated at different angles, or in any manner to enhance visual and measurement accuracy. Optionally, an alternate light source may replace the LED.

The switch 403 may be associated with an activator button which may preferably be located along the body 101, where it can easily be activated by one of the fingers of a hand that is holding the device. However it can be located in any other location on the body 101. The switch 403 may be selected from one or more: a coaxially mounted, a toggle switch, a depressible switch, or a shifting switch, an on-off switch, a momentary-on switch, a touch-activated switch using high gain field effect transistors, a depletion mode metal oxide semiconductor field effect transistor (MOSFET type) switch, a mechanical switch, a spring loaded switch or any type of switches which are generally simple devices and are well known in the art. A preferred embodiment of this invention is one in which the ovulation predictor device body optionally includes a mechanical actuator switch which is spring loaded and activated by removal of the protective cover 102. In this manner, a mechanical actuator may depress when a protective cover 102 of the device 100 is attached. Removal of the cover 102 by the user will allow the spring loaded switch mechanism to make contact and complete the light source circuit. This frees the user to examine the sample and focus the image.

With regard to the battery compartment 401, in one embodiment, the battery compartment 401 has a positive contact and a negative contact. The positive contact may be made of one or more of any material. One embodiment consists of one or more printed circuit boards 404 for one or more contacts 405. In one embodiment the contact 405 is made of copper. In one embodiment, the one end of the conducting strip may be formed to create a spring leaf which provides contact with the positive end of the battery terminal. In another embodiment, the other end of the conducting strip intimately contacts the activation switch 403. Preferably, one or more contact is a spring contact 405.

The one or more contacts 405 may be in continuous or non-continuous contact with the light 406 source. The one or more contacts 405 may be in continuous or non-continuous contact with leads 306 used to complete the circuit. The one or more printed circuit boards 404 may be in continuous or non-continuous contact with the lead 306. In one embodiment, the lead 306 is a wire, but the lead may be made of any conductive material known in the art. For example, the lead may be made form copper, tin, or nickel, or alternatively may be any material coated with a conductive material. In still another embodiment, the lead may be coated in non-conductive material except where it needs to be in contact with a power source, such as the batteries 408. The lead 306 may be through-hole or may be surface mounted. In another embodiment, the lead 306 is surface coated. In a preferred embodiment the lead 306 is not surface coated.

The positive and negative battery contacts 405 may be made of any conductive materials known to those skilled in the art, including, but not limited to copper, tin, or nickel. In a preferred embodiment, the battery contacts 405 are made from nickel. In yet another embodiment, the battery contact is coated with nickel. In another embodiment, the battery contacts 405 have a dimple or indentation or protrusion on the surface. The contact 405 may have a spring. In another embodiment, this dimple, indentation, protrusion or spring aids in wiping action and breaks any oxide coating that can occur over time on battery contact surfaces. In a preferred embodiment, the dimple, indentation, protrusion or spring provides sufficient surface contact force to break the oxide coating, without damaging the nickel or nickel coating. In a preferred embodiment, the surface contact force is in from about 400 to about 800 grams. In this way, the ovulation predictor device 100 has full and continuous power during use. This is a substantial improvement over other devices where corrosion in the battery compartment 401 reduces the power and the product life.

The battery compartment 401 may be made of any material presented herein. In one embodiment, it is made to accommodate button-type batteries 408 and preferably the entire electronic assembly 400. In another embodiment, battery compartment 401 is made of one or more injection molded parts. The battery compartment 401 may be inserted onto the any portion of the ovulation tester. In one embodiment it screws onto the base and is twisted to lock in place. In another embodiment, the battery compartment 401 may be connected to the ovulation predictor device 100 by any method known in the art, including, but not limited to snapping into place, strapping into place, or attaching by a hinged mechanism.

The ovulation predictor device 100 also has a battery contact 405 to alternately interrupt and permit the flow of current from the batteries 408, or other power source. In an alternative embodiment, the battery compartment 401 is eliminated, in favor of an external electrical source, known to those skilled in the art. For example, the user may hold the device up to a light source such as a light bulb from a lamp, mounted on a ceiling, or utilize ambient light.

Any power source may be utilized, including, but not limited to batteries 408, electricity or solar. In one embodiment, power harvesting techniques may be utilized including storage of mechanical actions converted to electrical energy by passing a permanent magnet through a conductive coil. The stored mechanical activation is mechanically produced by moving, shaking or cranking. In another embodiment, mechanical force is converted to electrical force utilizing the piezoelectric effect on certain crystals. In still another embodiment, the numerous electrical fields generated by power lines and radio transmitters is captured and stored to illuminate the ovulation prediction device 100. Energy may also be obtained by capturing time varying fields or physically passing the device through static fields to capture and store energy.

The base 402 may be placed anywhere in the ovulation predictor device and may have any type of switch 403 known in the art, including an on-off mechanical switch 406, a spring loaded switch 403, a momentary—on push button switch 403 and the like. Another embodiment of this invention is one in which the ovulation predictor device body 101 optionally includes a mechanical switch 403 which is spring loaded and activated by removal of a protective cover 102.

A preferred aspect of the invention is a method of predicting ovulation in an animal, utilizing the ovulation predictor device 100 described herein, said method comprising:
1. placing a specimen on the objective plate;
2. permitting the specimen to dry;
3. inserting said specimen into said ovulation predictor device;
4. activating the light switch; and
5. viewing the crystals produced from said dried specimen.

Although any bodily fluid can be utilized as a specimen for this method of detection, such as saliva, tears, urine, vaginal secretions or sweat, in a preferred embodiment, the specimen is a sample of saliva. In a preferred embodiment, the specimen is saliva. In another preferred embodiment, the animal is a human female.

FIG. 2. is a side exploded view of a preferred embodiment of the present invention. Shown is an ovulation predictor device 100, an ovulation predictor device body 101, and an optical subassembly 300. The optical subassembly is made of an objective mount 301, a focus ring 302, one or more lenses 303, an objective plate 304 and a focus ring lock pin 305.

In a preferred embodiment, the invention consists of a kit which contains the ovulation predictor device and instructions for use, and optionally a carrying case for the device.

### Electronic Imaging and Pattern Recognition

In one embodiment, the method includes displaying the results on a viewing screen, such as a computer monitor. In still another embodiment, the method may include storage of results once or over many times. In a preferred embodiment, the results may be compared to pre-determined norms derived from many other user samples. Furthermore, the results may be calculated by one or more algorithms, and optionally the results can be transferred from the ovulation device to a free standing computer or an electronically to a disc which can be transferred to a free standing computer.

In another embodiment, the method includes semiconductor substrates with alternating light emitting features and adjacent light detecting features. These may be arrayed linearly, in geometric patterns, or in specific patterns that allow electronic detection of the ferning pattern directly, and can be designed to promote or retard the growth of the ferning pattern. In still another embodiment, detection of the ferning pattern is performed by detecting light scattering, refracting, or polarization.

In yet another embodiment, the method includes detection of the physical properties of the crystals through impedance measurements. Further, detection of crystals or the hormones that trigger the crystals is accomplished by attaching chemical reagents and measuring the physical and optical properties thereof. Additionally, specific nuclear magnetic resonance or nuclear magnetic spectroscopy is performed to detect the crystal or the hormones triggering the crystal growth.

In still another embodiment, the kit optionally includes software program for monitoring and tracking ovulation, calculating results, and transferring them to stored or printed media.

In one embodiment, the ovulation predictor device contains an electronic imaging and pattern recognition device. In a preferred embodiment, the electronic imaging and pattern recognition device consists of one or more of a display, an electronic imaging plane, a processor that processes data.

In another embodiment the recognition device utilizes one or more algorithms capable of identifying transition in images from light to dark and of processing the signals in order to identify the presence of crystals and create a measurement of probability of ovulation.

The invention further consists of a method of mapping coordinates identified by the light and dark images wherein said method consists of one or more of the following: reading a planar image by said electronic imaging plane, processing the data through a first algorithm, identifying image transition from light to dark, mapping coordinates from said image, aligning the coordinates mathematically to create vectors (a second algorithm), comparing said vectors with predetermined mathematical descriptions of crystals; and determining the degree of correlation between the vectors and the image to determine the presence of crystals.

In still another embodiment, the method consists of utilizing a predictive software algorithm (fourth algorithm) which compares correlation factors over time and maps the data, and creates a measurement of the probability of ovulation. In another embodiment, the measurement of the probability of ovulation is achieved by first order derivative, comparing magnitudes of correlation values and the slopes of values. The electronic imaging and pattern recognition device may the results in one or more of the following: graphical, alphanumeric, color and monochromatic.

What is claimed:

1. A ovulation predictor device, comprising:
an ovulation predictor device body, an optical subassembly containing one or more aspheric lenses, an electronics assembly, a battery compartment, a light source and, optionally, a cover.

2. The ovulation predictor device of claim 1, wherein said optical subassembly comprises and objective mount, a focus ring, one or more aspheric lenses, an objective plate and a focus ring lock pin.

3. The ovulation predictor device of claim 1, wherein said electronics assembly comprises a battery compartment, a base, a switch, one or more printed circuit boards, one or more contacts, a light source, a resistor, a lead and one or more batteries.

4. The battery compartment of claim 3, wherein said battery compartment has a battery compartment opening for receiving the contents of the electronics assembly.

5. The ovulation predictor device of claim 1, wherein said body is tubular shaped.

6. The ovulation predictor device of claim 1, wherein the light source is a LED.

7. The ovulation predictor device of claim 1, wherein there are two aspheric lenses.

8. The ovulation predictor device of claim 1, wherein said one or more aspherical lenses enlarges the optical field and reduces aberration and blurring of the image, as compared to a spherical lens.

9. The ovulation predictor device of claim 1, wherein said one or more lenses form a focal length that is about 0.1 inches.

10. The ovulation predictor device of claim 1, wherein said focus ring has a focus ring top which comprises a magnifying lens.

11. The ovulation predictor device of claim 1, wherein said electronics assembly comprises one or more contacts.

12. The ovulation predictor device of claim 11, wherein said contacts are manufactured from nickel.

13. The ovulation predictor kit of claim 11, wherein said contacts have dimples, indentations, protrusions or springs.

14. The ovulation predictor device of claim 1, further comprising an alternative power source.

15. The ovulation predictor device of claim 14, wherein said alternative power source is selected from:
a. batteries
b. rechargeable batteries;
c. solar panels;
d. piezoelectric;
e. storage of mechanical actions converted to electrical energy by passing a magnet through a conductive coil, said mechanical activation by moving, shaking or cranking;
f. harvesting power from fields generated by power lines or radio transmitters; and
g. capturing time varying fields or physically passing the device through static fields to capture and store energy.

16. The LED of claim 5, wherein said LED emits a wavelength of light in the visible spectrum.

17. The LED of claim 15, wherein said LED emits a green light.

18. A kit comprising the ovulation predictor device of claim 1, instructions on how to use the device and, optionally a carrying case.

19. A kit comprising the ovulation predictor device of claim 1, instructions on how to use the device, a multi-language book with instructions, an educational interview on compact disc, a discrete carrying case, a microfiber cleaning cloth and a fertility chart.

20. A method of predicting ovulation in an animal, utilizing the ovulation predictor device of claim 1, said method comprising:
a. placing a specimen on the objective plate;
b. permitting the specimen to dry;
c. inserting said specimen into said ovulation predictor device;
d. activating the light switch; and
e. viewing the crystals produced from said dried specimen.

21. The method of claim 20 wherein said animal is a human.

22. The method of claim 20 wherein said specimen is saliva.

23. A method of determining whether a pregnant female has premature rupture of the membranes (PROM).

24. The ovulation predictor device of claim 1, further comprising a battery compartment that is capable of providing long-term and reliable power by eliminating galvanic corrosion to the battery contacts.

25. The ovulation predictor device of claim 1, further comprising an electronic imaging and pattern recognition device.

26. The electronic imaging and pattern recognition device of claim 24, comprising:
a. a display;
b. an electronic imaging plane;
c. a processor that processes data;
d. one or more algorithms capable of identifying transition in images from light to dark and of processing the signals in order to identify the presence of crystals and create a measurement of probability of ovulation.

27. A method of mapping coordinates wherein said method comprises:
a. reading a planar image by said electronic imaging plane;
b. processing the data through a first algorithm;
c. identifying image transition from light to dark
d. mapping coordinates from said image;
e. aligning the coordinates mathematically to create vectors (a second algorithm);
f. comparing said vectors with predetermined mathematical descriptions of crystals; and
g. determining the degree of correlation between the vectors and the image to determine the presence of crystals.

28. The method of claim 26 wherein a predictive software algorithm (fourth algorithm) compares correlation factors over time and maps the data, and creates a measurement of the probability of ovulation.

29. The method of claim 27 wherein said measurement of the probability of ovulation is achieved by first order derivative, comparing magnitudes of correlation values and the slopes of values.

30. The electronic imaging and pattern recognition device of claim 24, wherein said display is selected from graphical, alphanumeric, color and monochromatic.

31. The method of claim 24 wherein results are displayed on a viewing screen.

32. The method of claim 24 wherein the results are calculated by one or more algorithms.

33. The method of claim 24 wherein the results can be transferred from the ovulation device to a free standing computer.

34. The ovulation predictor device of claim 1, further comprising an electronic disc which can be transferred to a free standing computer.

35. The kit of claim 31 comprising one or more software programs to assist the user in tracking and determining ovulation.

36. The ovulation predictor device of claim 1, further comprising a semiconductor substrate with alternating light producing and light detecting elements.

37. The semiconductor substrate with alternating light producing and light detecting elements of claim 36, comprising:
a. a linear pattern;
b. a geometric pattern;
c. a specific pattern to detect the ferning pattern directly.

38. The ovulation predictor device of claim 1, further comprising a sensor for detecting:
a. scattered light from the crystals;
b. refracted light from the crystals;
c. polarized light from the crystals;
d. impedance of the crystals;
e. physical and optical properties of attached chemical reagents;
f. nuclear magnetic properties of the crystals.
